(19) United States
(12) Patent Application Publication
(10) Pub. No.: US 2025/0281452 A1
(43) Pub. Date: Sep. 11, 2025

(54) CHIRAL ARYL PROPIONIC ACID DERIVATIVE AND PHARMACEUTICAL COMPOSITION THEREOF, AND USE

(71) Applicant: SHIJIAZHUANG DISCOVERY MEDICINE TECHNOLOGY CO., LTD, Shijiazhuang , Hebei (CN)

(72) Inventors: Yuelei MENG, Shijiazhuang (CN); Lu WANG, Shijiazhuang (CN); Liang GAO, Shijiazhuang (CN)

(73) Assignee: SHIJIAZHUANG DISCOVERY MEDICINE TECHNOLOGY CO., LTD, Shijiazhuang , Hebei (CN)

(21) Appl. No.: 18/858,534

(22) PCT Filed: Apr. 18, 2023
(86) PCT No.: PCT/CN2023/088864

§ 371 Date: Oct. 21, 2024

(30) Foreign Application Priority Data
Apr. 19, 2022 (CN) 202210408886.8

(51) Int. Cl. A61K 31/40; A61K 31/216; A61K 31/222; A61K 31/223; C07C 217/10; C07C 219/10; C07C 229/12; C07D 207/404

(52) U.S. Cl. CPC A61K 31/40; A61K 31/216; A61K 31/222; A61K 31/223; C07C 217/10; C07C 219/10; C07C 229/12; C07D 207/404

(57) ABSTRACT

A chiral aryl propionic acid derivative and a pharmaceutical composition thereof, and a use. The chiral aryl propionic acid derivative is as shown in formula (I). The chiral aryl propionic acid derivative has antipyretic, analgesic and anti-inflammatory effects. The compound has high activity and can be locally administrated, a reduced dosage is expected, and adverse reactions are reduced.

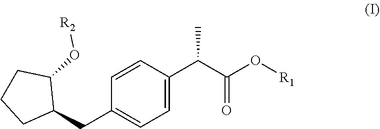

### TECHNICAL FIELD

The present invention relates to but is not limited to the technical field of pharmaceutical chemistry, in particular to a chiral aryl propionic acid derivative and a pharmaceutical composition thereof, and a use.

### BACKGROUND

Loxoprofen sodium with a chemical name of sodium 2-[4-(2-oxocyclopentyl-1-ylmethyl)phenyl] propionate is the first propionic acid precursor non-steroidal anti-inflammatory drugs (NSAIDs), which is developed by Japan Sankyo Corporation. Loxoprofen Sodium Tablets were launched in Japan in July 1986 under the trade name Loxonin, and marketed in China in 1999 under the original Chinese trademark name “Le Song”. The dosage forms of the marketed drugs are tablets, capsules, fine particles, patches and gels. Among various dosage forms of marketed drugs, the API is present in a form of loxoprofen sodium dihydrate.

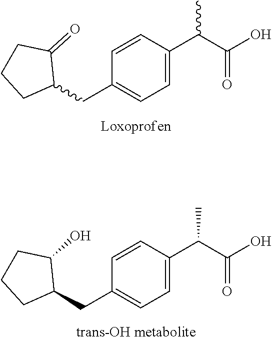 [SMILES: CC(C(=O)O)c1ccc(CC2CCCC2=O)cc1 ; C[C@H](C(=O)O)c1ccc(C[C@H]2CCC[C@@H]2O)cc1]

Loxoprofen is a prodrug, which has weak activity and is metabolized by liver after oral administration to transform loxoprofen into an active ingredient so as to exert its therapeutic effect. Loxoprofen is considered as being metabolized by a carbonyl reductase in skins and subcutaneous muscle tissues after local administration. Loxoprofen has 2 chiral centers and 4 chiral isomers. At present, loxoprofen sodium is marketed in a racemic form. There are no literatures reporting differences in pharmacological effects among four isomers of loxoprofen. After being orally administrated, loxoprofen is rapidly absorbed. After 30-50 minutes, the plasma concentrations of loxoprofen and its metabolic products reach peaks, and plasma protein binding rates are 97% and 93%, respectively; after single local administration of 1% loxoprofen (100 mg), 10% of dose is transferred to a body in 12 hours.

Carbonyl is reduced to hydroxyl upon loxoprofen is metabolized, and 3 chiral centers and 8 isomers will be comprised in the structure at this point, among them a trans-OH metabolite is one of the metabolites. For in-vivo metabolites containing 3 chiral centers, there are no literatures reporting the differences in specific pharmacological effects of the corresponding diastereoisomers.

Detailed researches have been conducted on several isomers, and an efficient fast-acting non-steroidal anti-inflammatory drug derivative has been developed through structural modifications on this basis.

### SUMMARY

The inventors develop a chiral aryl propionic acid derivative. The compound has antipyretic, analgesic and anti-inflammatory effects. The compound of the present invention has a better anti-rheumatoid arthritis effect, and it is expected to reduce the administration dose and lower adverse reactions. In addition, it is unexpectedly found that the chiral aryl propionic acid derivative is highly distributed in joints. Further studies have shown that when being externally applied to skins, the chiral aryl propionic acid derivative has better joint fluid distribution and better therapeutic effects, and can be used as a locally administrated non-steroidal anti-inflammatory drug.

One aspect of the present invention provides a chiral aryl propionic acid derivative as shown in formula (I), and a tautomer, a solvate or a pharmaceutically acceptable salt thereof:

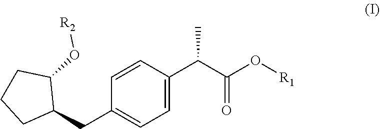

in the formula (I), R₁ is H, or is selected from

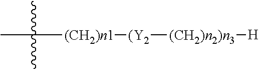

substituted by one or more

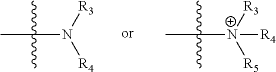

or unsubstituted

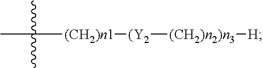

R₂ is H, or is selected from

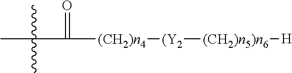

substituted by one or more

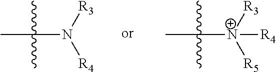

or unsubstituted

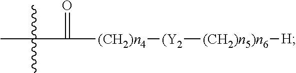

and when R₁ is H, R₂ is not H;
Y₂ is O, or N(R₆), or S;
n₁, n₂, n₃, n₄, n₅ and n₆ are each independently 0, 1, 2, 3, 4, 5, 6, 7, 8, 9, and 10;
R₃, R₄ and R₅ are each independently H, C1-C20 hydrocarbyl, or C1-C20 alkyl carbonyl, or R₃ and R₄ form a ring together with a nitrogen atom to which they are attached;
R₆ is H, or C1-C20 hydrocarbyl;
A⁻ represents an acceptable inorganic or organic anion.

In the embodiments of the present application, the hydrocarbyl comprises an alkyl hydrocarbyl, an alkenyl hydrocarbyl, or an alkynyl hydrocarbyl, and also comprises a heterocyclyl, an aryl, or a heteroaryl.

In the embodiments of the present application, the hydrocarbyl comprises a straight, branched or cyclic hydrocarbyl.

In the embodiments of the present invention, the C1-C20 hydrocarbyl refers to a saturated or unsaturated aliphatic hydrocarbyl containing 1 to 20 carbon atoms, including C1-C20 alkyl hydrocarbyl, C2-C20 alkenyl hydrocarbyl, and C2-C20 alkynyl hydrocarbyl. For example, the C1-C20 hydrocarbyl includes but is not limited to methyl, ethyl, propyl, isopropyl, butyl, sec-butyl, tert-butyl, pentyl, hexyl, cyclopropyl, cyclobutyl, cyclopentyl, cyclohexyl, ethenyl, ethynyl, propenyl, propynyl, allyl, 2-methyl-2-butenyl, 2-butenyl (—CH₂—CH═CH—CH₃), 3-butenyl (—CH₂—CH₂—CH═CH₂), 4-pentenyl (—CH₂—CH₂—CH₂—CH═CH₂), 2-methyl-2-pentenyl (—CH₂—C(CH₃)═CH—CH₂—CH₃) and 5-hexenyl (—CH₂CH₂CH₂CH═CH₂).

In the embodiments of the present application, the hydrocarbyl comprises a C2-C20 heterocyclyl. The heterocyclyl means that this heterocycle at least comprises one or more atoms selected from oxygen, nitrogen and sulfur; and includes, but is not limited to: oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidyl, piperazinyl, isoquinolyl, tetrahydroisoquinolyl, morpholinyl, thiomorpholinyl, 1,1-dioxo-thiomorpholinyl, dihydropyranyl, tetrahydropyranyl, hexahydrothiapyranyl, hexahydropyrimidinyl, oxazacyclohexyl, thiazanyl, thioxanyl, homopiperazinyl, homopiperidyl, azepanyl, oxepanyl, thiepanyl, oxazepanyl, diazepanyl, 1,4-diazepanyl, thiazetanyl, tetrahydro thiapyranyl, oxazolidinyl, thiazolidinyl, isothiazolidinyl, 1,1-dioxoisothiazolidinonyl and oxazolidinonyl.

In the embodiments of the present application, the hydrocarbyl comprises aryl including but not limited to: benzene, naphthalene, anthracene, or biphenyl, etc.

In the embodiments of the present application, the hydrocarbyl comprises heteroaryl including but not limited to: pyrimidine, furan, thiazole, thiophene, pyridine, pyrrole and imidazole.

In the embodiments of the present application, the pharmaceutically acceptable salt refers to any form of chiral aryl propionic acid derivative used according to the present invention, wherein the chiral aryl propionic acid derivative is in an ion form or charged, and is coupled with counter ions (negative ions or positive ions) or is in a solution;

In the embodiments of the present application, the pharmaceutically acceptable salt comprises salts formed by the chiral aryl propionic acid derivative of the present invention and negative ions, the negative ions include but are not limited to: fluoride ions, chloride ions, bromide ions, iodide ions, acetate ions, benzoate ions, citrate ions, tartrate ions, oxalate ions, malate ions, ascorbate ions, and fumarate ions, etc.;

In the embodiments of the present application, the pharmaceutically acceptable salt includes an inner salt formed by the chiral aryl propionic acid derivative in the present invention, and the inner salt refers to a salt formed within a molecule when the molecule contains both carboxyl and amino, indicating that the same molecule carries both positive and negative charges;

In the embodiments of the present application, the pharmaceutically acceptable salt comprises a salt formed between the molecules of the chiral aryl propionic acid derivative of the present invention. The salt formed between the molecules refers to a salt formed between different molecules when the molecule of the chiral aryl propionic acid derivative contains both carboxyl and amino.

In some embodiments, R₁ is hydrogen, and R₂ is

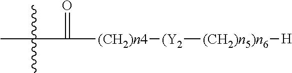

substituted by one or more

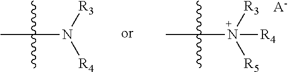

or unsubstituted

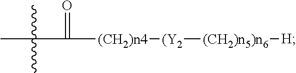

In some embodiments, R₂ is hydrogen, and R₁ is

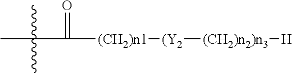

substituted by one or more

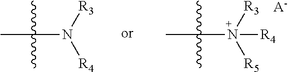

or unsubstituted

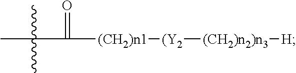

In some embodiments, R₁ is

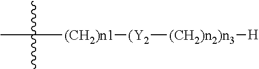

substituted by one or more

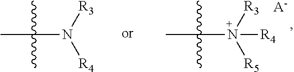

### and R₂ is

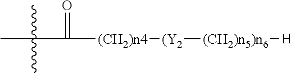

substituted by one or more

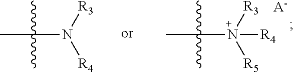

In some embodiments, R₁ is

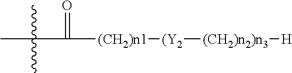

substituted or unsubstituted by one or more

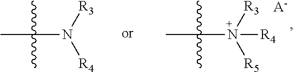

### and R₂ is

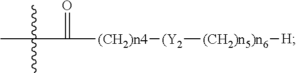

In some embodiments, R₁ is

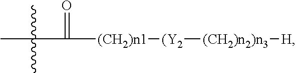

### and R₂ is

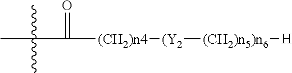

substituted by one or more

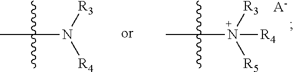

In some embodiments, when R₁ or R₂ is not hydrogen, its substituent

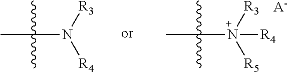

may replace the hydrogen on the terminal carbon, or the hydrogen on the non-terminal carbon.

In some embodiments, R₃ and R₄ are both H;

In some embodiments, R₃ and R₄ are both C1-C20 hydrocarbyl, or C1-C20 alkyl carbonyl; preferably, R₃ and R₄ are both C1-C6 hydrocarbyl, or C1-C6 alkyl carbonyl; more preferably, R₃ and R₄ are both C1-C6 alkyl;

In some embodiments, R₃ is H, and R₄ is C1-C20 hydrocarbyl, or C1-C20 alkyl carbonyl; preferably, R₄ is both C1-C6 hydrocarbyl; more preferably, R₄ is C1-C6 alkyl;

In some embodiments, R₄ is H, and R₃ is C1-C20 hydrocarbyl, or C1-C20 alkyl carbonyl; preferably, R₃ is both C1-C6 hydrocarbyl; more preferably, R₃ is C1-C6 alkyl;

In some embodiments, R₃ and R₄ form a ring together with a nitrogen atom to which they are attached, wherein the nitrogen atom can be attached to R₃ and/or R₄ via a common carbon-nitrogen bond, or via an amide bond; the ring formed by R₃ and R₄ together with the nitrogen atom to which they are attached can be a 4-membered ring, a 5-membered ring, a 6-membered ring, or a 7-membered ring.

In some embodiments, R₃, R₄ and R₅ are all H;

In some embodiments, R₃ and R₄ are both C1-C20 hydrocarbyl or C1-C20 alkyl carbonyl, and R₅ is H; preferably, R₃ and R₄ are both C1-C6 hydrocarbyl or C1-C6 alkyl carbonyl, and R₅ is H; more preferably, R₃ and R₄ are both C1-C6 alkyl, and R₅ is H;

In some embodiments, R₃, R₄ and R₅ are all C1-C20 hydrocarbyl or C1-C20 alkyl carbonyl; preferably, R₃, R₄ and R₅ are all C1-C6 hydrocarbyl; more preferably, R₃, R₄ and R₅ are all C1-C6 alkyl;

In some embodiments, R₃ and R₄ form a ring together with a nitrogen atom to which they are attached, and R₅ is both H or C1-C20 hydrocarbyl, wherein, the nitrogen atom and R₃ and/or R₄ can be attached via a common carbon-nitrogen bond, or via an amide bond; the ring formed by R₃ and R₄ together with the nitrogen atom to which they are attached can be a 4-membered ring, a 5-membered ring, a 6-membered ring, or a 7-membered ring.

In some embodiments, Y₂ is O, or N(R₆), or S;

In some embodiments, Y₂ is N(R₆), or S.

In some embodiments, Y₂ is N(R₆), or O.

In some embodiments, Y₂ is O, or N(R₆), or S.

In some embodiments, R₆ is H.

In some embodiments, R₆ is C1-C20 hydrocarbyl; preferably, R₆ is C1-C6 hydrocarbyl; more preferably, R₆ is C1-C6 alkyl.

In some embodiments, n₁, n₂ and n₃ are each independently 0, or 1, or 2, or 3, or 4, or 5, or 6, or 7, or 8, or 9, or 10, preferably, n₁, n₂ and n₃ are each independently 0, or 1, or 2, or 3, or 4;

In some embodiments, n₁, n₂ and n₃ are all 0.

In some embodiments, n₄, n₅ and n₆ are each independently 0, or 1, or 2, or 3, or 4, or 5, or 6, or 7, or 8, or 9, or 10, preferably, n₁, n₂ and n₃ are each independently 0, or 1, or 2, or 3, or 4;

In some embodiments, n₄, n₅ and n₆ are all 0.

In some embodiments, A⁻ is selected from the group consisting of halide ions, perchlorate, nitrate, sulfate, sulfhydrate, sulfite, phosphate, hydrophosphate, C1-C8 alkyl acid radicals, C1-C8 alkyl sulfonate, C1-C8 alkyl sulfate, and C1-C8 aryl sulfonate;

In some more specific embodiments, A⁻ is preferably selected from sulfate, phosphate, acetate, propionate, chloride and bromide ions.

In some embodiments, when R₁ is hydrogen and R₂ is

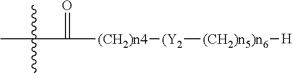

substituted by one or more

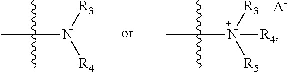

the chiral aryl propionic acid derivative of the present invention can form an inner salt or an intermolecular salt under proper conditions;

In some embodiments, when R₁ is hydrogen and R₂ is

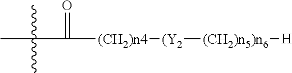

substituted by one or more

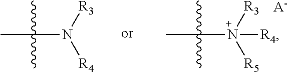

the chiral aryl propionic acid derivative of the present invention can form salts with other anions under proper conditions, and the anions are as described above.

In some embodiments, when R₂ is hydrogen and R₁ is

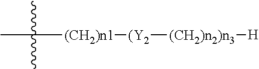

substituted by one or more

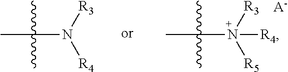

the chiral aryl propionic acid derivative of the present invention can form salts with other anions under proper conditions, and the anions are as described above;

In some embodiments, when R₁ is

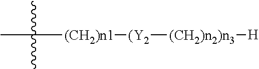

substituted by one or more

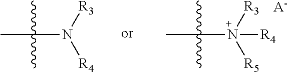

### and R₂ is

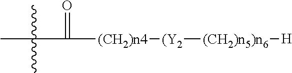

substituted by one or more

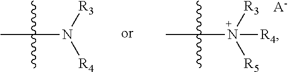

the chiral aryl propionic acid derivative of the present invention can form salts with other anions under proper conditions, and the anions are as described above.

In some embodiments, the chiral aryl propionic acid derivative provided in the present invention is selected from the following compounds:

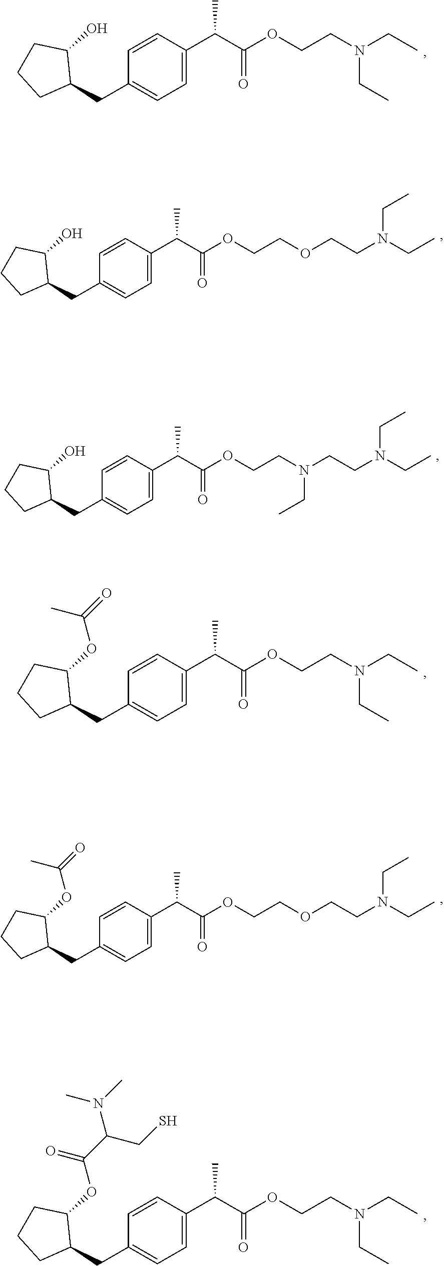 [SMILES: CCN(CC)CCOC(=O)[C@@H](C)c1ccc(C[C@H]2CCC[C@@H]2O)cc1 ; CCN(CC)CCOCCOC(=O)[C@@H](C)c1ccc(C[C@H]2CCC[C@@H]2O)cc1 ; CCN(CC)CCN(CC)CCOC(=O)[C@@H](C)c1ccc(C[C@H]2CCC[C@@H]2O)cc1 ; CCN(CC)CCOC(=O)[C@@H](C)c1ccc(C[C@H]2CCC[C@@H]2OC(C)=O)cc1 ; CCN(CC)CCOCCOC(=O)[C@@H](C)c1ccc(C[C@H]2CCC[C@@H]2OC(C)=O)cc1 ; CCN(CC)CCOC(=O)[C@@H](C)c1ccc(C[C@H]2CCC[C@@H]2OC(=O)C(CS)N(C)C)cc1]

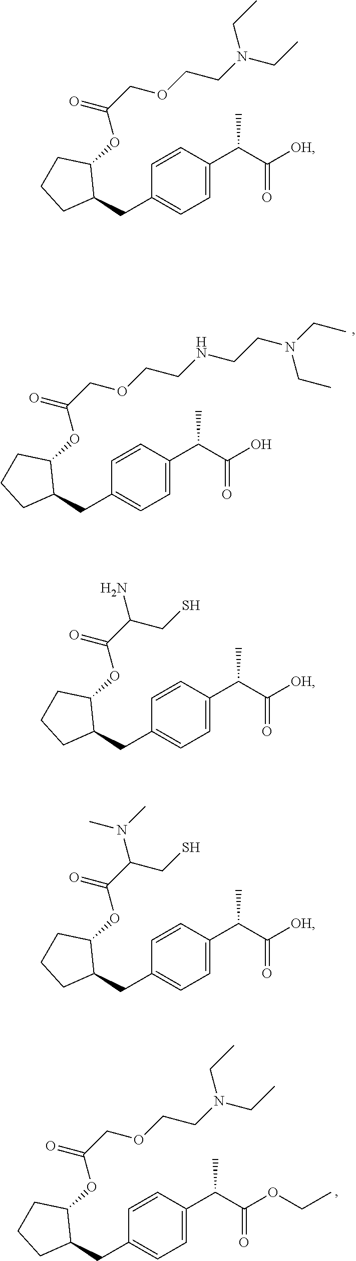 [SMILES: CCN(CC)CCOCC(=O)O[C@H]1CCC[C@@H]1Cc1ccc([C@H](C)C(=O)O)cc1 ; CCN(CC)CCNCCOCC(=O)O[C@H]1CCC[C@@H]1Cc1ccc([C@H](C)C(=O)O)cc1 ; C[C@H](C(=O)O)c1ccc(C[C@H]2CCC[C@@H]2OC(=O)C(N)CS)cc1 ; C[C@H](C(=O)O)c1ccc(C[C@H]2CCC[C@@H]2OC(=O)C(CS)N(C)C)cc1 ; CCOC(=O)[C@@H](C)c1ccc(C[C@H]2CCC[C@@H]2OC(=O)COCCN(CC)CC)cc1]

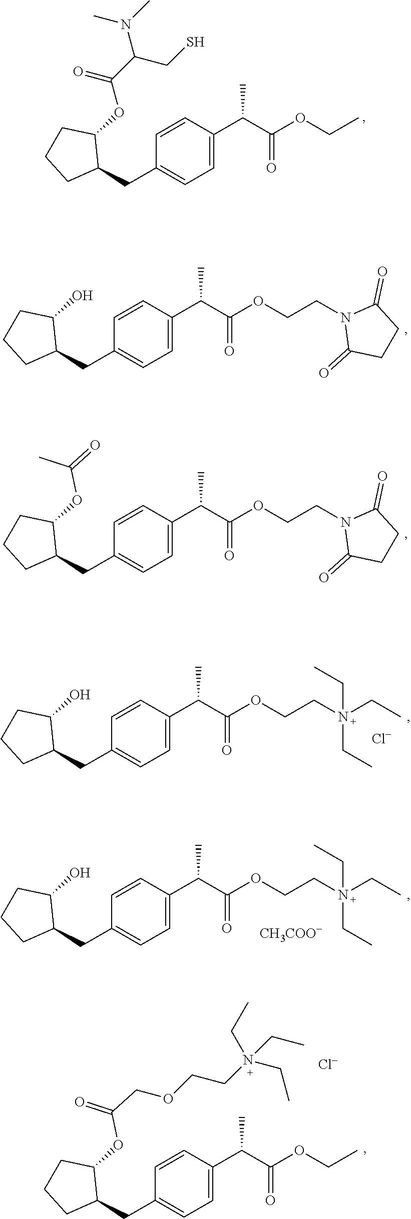

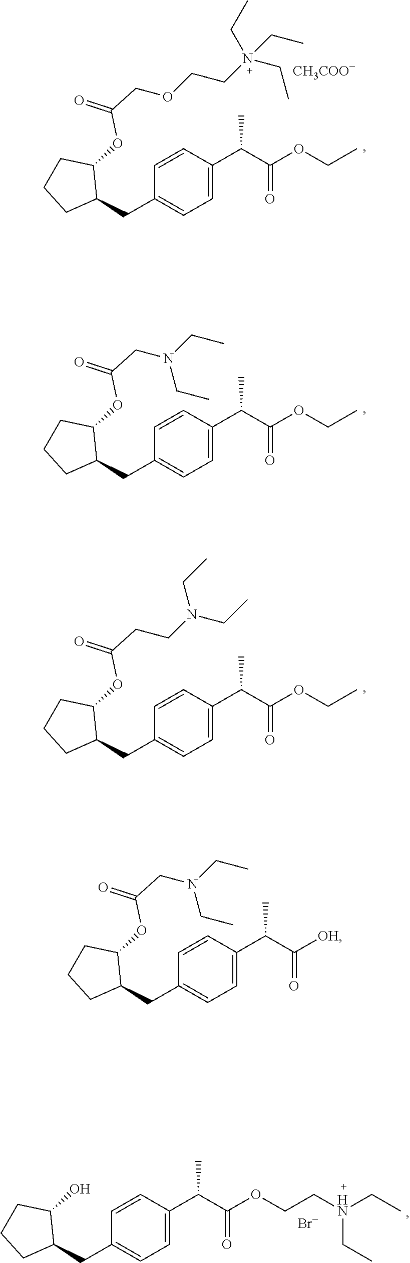

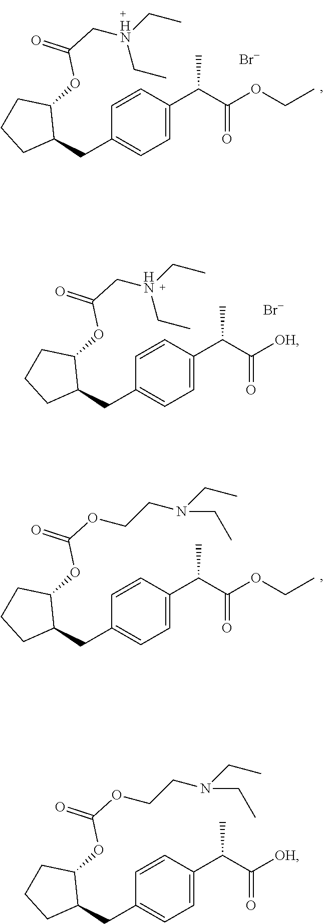

or a pharmaceutically acceptable salt thereof.

In another aspect, the present invention provides a pharmaceutical composition comprising the above mentioned chiral aryl propionic acid derivative, a tautomer, a solvate or a pharmaceutically acceptable salt thereof.

The present invention discloses a pharmaceutical composition which is composed of the chiral aryl propionic acid derivative, a tautomer, a solvate or a pharmaceutically acceptable salt thereof as described in the present invention as an active ingredient or a main active ingredient in combination with a pharmaceutically acceptable carrier.

The chiral aryl propionic acid derivative of the present invention can be prepared into a pharmaceutical composition. The prepared pharmaceutical composition is administrated to a patient in multiple properly selected administration routes including gastrointestinal local administration, for example, the chiral aryl propionic acid derivative can be prepared into topical preparations for skins, ophthalmic preparations, inhalation preparations and the like.

In some examples of the present invention, the chiral aryl propionic acid derivative of the present invention and lactose are mixed and smashed, and therefore prepared into inhalants.

In some examples of the present invention, the chiral aryl propionic acid derivative of the present invention and a proper amount of surfactants and osmotic pressure regulators are jointly dissolved and then prepared into a solution for inhalation.

In some examples of the present invention, the chiral aryl propionic acid derivative of the present invention a proper amount of surfactants and the like are jointly prepared into topical preparations for skins.

In some examples of the present invention, the chiral aryl propionic acid derivative of the present invention and proper accessories and the like are jointly prepared into ophthalmic preparations.

In a third aspect, the compound of the present invention has high bioavailability when it is administrated gastroenterally/parenterally and can be rapidly transformed into an active ingredient, and especially when it is administrated endermically or orally, it has a better effect.

The compound of the present invention has a better anti-rheumatoid arthritis effect. The compound of the present invention has a significant inhibitory effect on the proliferation of INF-α induced human rheumatoid arthritis fibroblast-like synovial cells (HFLS-RA), and can significantly inhibit the expression of inflammatory cytokines, exhibiting higher activity, and it is expected to reduce the administration dose and lower adverse reactions.

In addition, it is unexpectedly found that the compound of the present invention is highly distributed at joints after being orally administrated. The further study shows that the compound of the present invention has better joint fluid distribution and better therapeutic effects when externally applied on the skin, and can be used as a locally administrated non-steroidal anti-inflammatory drug.

In a fourth aspect, the present invention provides use of the above mentioned chiral aryl propionic acid derivative, a tautomer, a solvate or a pharmaceutically acceptable salt thereof as a non-steroidal anti-inflammatory drug, which is mainly useful for anti-inflammatory and analgesic treatment of conditions such as arthritis, rheumatoid arthritis, lumbago, scapulohumeral periarthritis and neck-shoulder-wrist syndrome, etc., as well as postoperative anti-inflammatory and analgesic treatment and antipyretic and analgesic treatment for acute respiratory inflammation.

### BRIEF DESCRIPTION OF THE DRAWINGS

FIG. 1 shows the comparison of distribution of control 1, control 2 and compounds DSC4813 and DSC4821 of the present invention at joints.

### DETAILED DESCRIPTION

The present invention can be more comprehensively understood by those skilled in the art through the following examples, which will not limit the present invention in any ways. The structures of all the compounds are determined by MS or ¹H NMR.

### Example 1: Synthesis of Trans-OH Metabolite

A compound trans-OH metabolite was synthesized with reference to the literature (Mandai, T. and T. Yamakawa (2000). “An Efficient Synthesis of (2S)-2-[4-((1R,2S)-2-Hydroxycyclopentylmethyl)phenyl] propionic Acid.” Synlett 2000(06): 0862-0864), mp 87-88° C., [M−H]⁻=247.16. ¹H NMR (300 MHz, CDCl₃) δ: 1.20-1.32 (m, 1H), 1.41-1.82 (m, 7H), 1.88-2.06 (m, 2H), 2.46 (dd, 1H), 2.75 (dd, 1H), 3.69 (q, 1H), 3.86-3.95 (m, 1H), 7.10-7.24 (m, 4H).

### Example 2: Synthesis of DSC4801

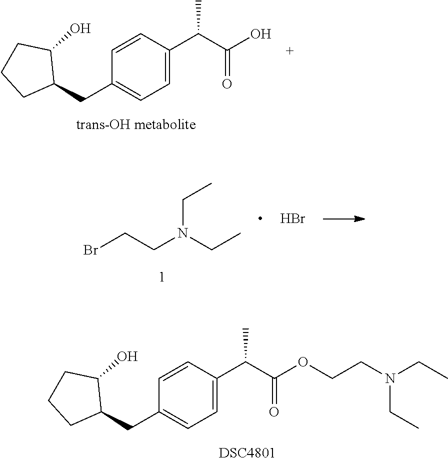

0.8 g of compound trans-OH metabolite was added into 10 mL of N,N-dimethylformamide (DMF), the system was cooled to 0° C., 1.01 g of compound 1 (2-bromo-N,N-diethylethylamine hydrobromide) and 0.85 g of sodium carbonate were added thereto, the system was stirred for 18 h. Water was added to precipitate out a solid, the solid was filtrated and then added into 20 mL of dichloromethane, water was added for liquid separation, the organic phase was concentrated to dryness, and then purified via a silica gel column to obtain 0.72 g of compound DSC4801, with a yield of 64%, [M+H]⁺=348.33, ¹H NMR (300 MHz, CDCl₃) δ:0.95 (t, 6H), 1.19-1.30 (m, 1H), 1.41-1.71 (m, 6H), 1.75-1.85 (m, 1H), 1.94-2.14 (m, 2H), 2.44 (dd, 1H), 2.51 (m, 4H), 2.78 (m, 3H), 3.74 (q, 1H), 3.86-4.05 (m, 1H), 4.26 (t, 2H), 7.11-7.21 (m, 4H).

### Example 3: Synthesis of DSC4804

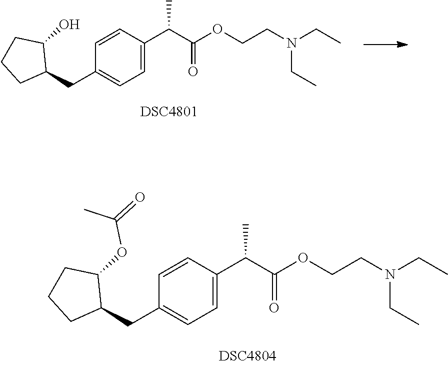 [SMILES: CCN(CC)CCOC(=O)[C@@H](C)c1ccc(C[C@H]2CCC[C@@H]2O)cc1 ; CCN(CC)CCOC(=O)[C@@H](C)c1ccc(C[C@H]2CCC[C@@H]2OC(C)=O)cc1]

0.45 g of compound DSC4801, 0.15 g of pyridine and 10 mL of acetonitrile were added into a reaction flask, the reaction mixture was heated to 35° C., then 0.2 g of acetic anhydride was added to the above reaction mixture at 35-40° C., and then the mixture was reacted for 4 h at 50° C., the system was concentrated to dryness and then cooled to room temperature, 15 mL of dichloromethane and 15 mL of water were added into the system for liquid separation, and the organic phase was concentrated to dryness, and then purified via a silica gel column to obtain 0.39 g of compound DSC4804, with a yield of 77%; [M+H]⁺=390.19, ¹H NMR (300 MHz, CDCl₃) δ:1.08 (t, 6H), 1.17-1.30 (m, 1H), 1.45-1.86 (m, 7H), 1.94-2.11 (m, 5H), 2.49 (dd, 1H), 2.53 (m, 4H), 2.75 (m, 3H), 3.76 (q, 1H), 3.81-3.98 (m, 1H), 4.3 (t, 2H), 7.07-7.20 (m, 4H).

### Example 4: Synthesis of DSC4807

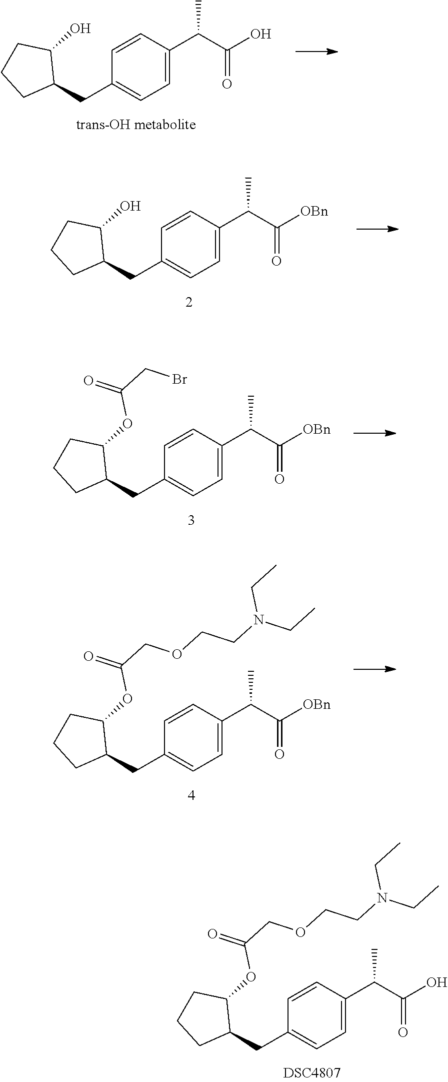 [SMILES: C[C@H](C(=O)O)c1ccc(C[C@H]2CCC[C@@H]2O)cc1 ; C[C@H](C(=O)OCc1ccccc1)c1ccc(C[C@H]2CCC[C@@H]2O)cc1 ; C[C@H](C(=O)OCc1ccccc1)c1ccc(C[C@H]2CCC[C@@H]2OC(=O)CBr)cc1 ; CCN(CC)CCOCC(=O)O[C@H]1CCC[C@@H]1Cc1ccc([C@H](C)C(=O)OCc2ccccc2)cc1 ; CCN(CC)CCOCC(=O)O[C@H]1CCC[C@@H]1Cc1ccc([C@H](C)C(=O)O)cc1]

### Synthesis of Compound 2

1.0 g of compound trans-OH metabolite was added into 10 mL of N,N-dimethylformamide (DMF), the system was cooled to 0° C., 0.83 g of benzyl bromide and 0.55 g of sodium carbonate were added thereto, the system was stirred for 3 h. Water was added to the system to precipitate out a solid, the solid was filtered and then added into 20 mL of dimethylformamide, then water was added for liquid separation, and the organic phase was concentrated to dryness, and then purified via a silica gel column to obtain 1.12 g of compound 2, with a yield of 82%; [M+H]⁺=339.10;

### Synthesis of Compound 3

1.0 g of compound 2, 0.3 g of pyridine and 20 mL of anhydrous dichloromethane were added into a reaction flask, the reaction mixture was cooled to 0° C., then 0.66 g of bromoacetyl bromide was added, the reaction system was reacted for 12 h at room temperature, water was added into the above system for liquid separation, the organic phase was concentrated to dryness, and then purified via a silica gel column to obtain 1.06 g of compound 3, with a yield of 78%; [M+H]⁺=459.21;

### Synthesis of Compound 4

0.2 g of compound diethylaminoethanol and 15 mL of anhydrous tetrahydrofuran were added into a reaction flask, the system was cooled to 0° C., and 0.05 g of sodium hydride (60%) was added, and then the system was stirred for 30 minutes. 0.71 g of compound 3 was added, the reaction mixture was heated to 35° C. to react for 4 h, the above system was concentrated to dryness and cooled to room temperature, 15 mL of dichloromethane and 15 mL of water were added for liquid separation, the organic phase was concentrated to dryness, and then purified via a silica gel column to obtain 0.63 g of compound 4, with a yield of 75%; [M+H]⁺=496.34;

### Synthesis of Compound DSC4807

1.19 g of 10% palladium carbon was added into 0.5 g of compound 4, 10 mL of methanol was added into the above system, then the system was connected with a hydrogen balloon, and heated to 30° C. to react for 8 h. The system was filtered, the filtrate was concentrated to dryness, and then purified via a silica gel column to obtain 0.36 g of compound DSC4807, with a yield of 88%; [M−H]⁻=404.29.

### Example 5: Synthesis of DSC4806

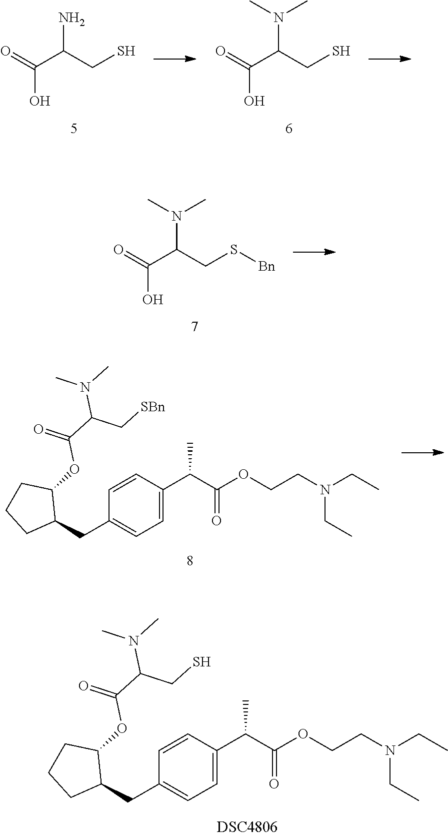

### Synthesis of Compound 6

Compound 6 was synthesized with reference to the method in the literature (“Characterization of N,N-dimethyl amino acids by electrospray ionization-tandem mass spectrometry.” J. Mass Spectrom. 2015, 50, 771-781.

### Synthesis of Compound 7

1.2 g of compound 6 and 30 mL of ethanol were added into a reaction flask, then 10 mL of 2N sodium hydroxide solution was added into the above system, and then 1.51 g of benzyl bromide was added with intense stirring. After the system was reacted for 2 h at room temperature, concentrated hydrochloric acid was added to adjust pH to be neutral, a solid was precipitated out and filtered, the filter cake was washed with water and ethanol successively, and the system was dried to obtain 1.48 g of compound 7, with a yield of 77%, [M−H]⁻=238.25.

### Synthesis of Compound 8

1.2 g of compound 7 and 15 mL of dichloromethane were added into a reaction flask, 1 g of thionyl chloride was added into the above system, then the mixture was reacted for 3 h at room temperature, the system was concentrated to dryness, and then 30 mL of dichloromethane was added again, and then the system was concentrated to dryness. 15 mL of dichloromethane and 1 mL of triethylamine were added into the system, 1.74 g of compound DSC4801 was added thereto, the system was heated to 40° C. to react for 5 h, and then it was concentrated to dryness, and then purified via a silica gel column to obtain 1.83 g of compound 8, with a yield of 64%, [M+H]⁺=569.33;

### Synthesis of Compound DSC4806

0.25 g of 5% palladium/carbon was added into 0.6 g of compound 8, 10 mL of methanol was added into the above system, then the system was connected with a hydrogen balloon, and then the system was heated to 30° C. to react for 8 h. The system was filtered, and the filtrate was concentrated to dryness, and then purified via a silica gel column to obtain 0.43 g of compound DSC4806, with a yield of 85%; [M+H]⁺=479.29.

### Example 6: Synthesis of DSC4815 and DSC4816

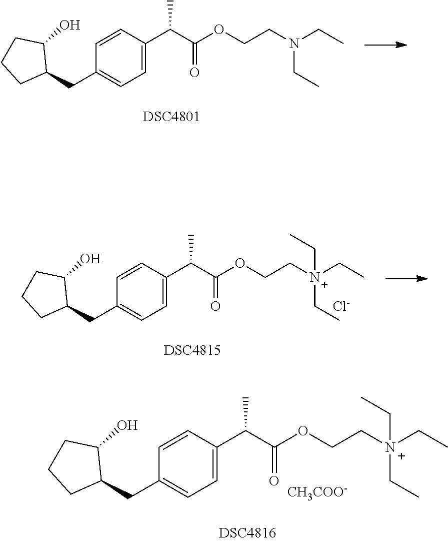

### Synthesis of Compound DSC4815

20 mL of tetrahydrofuran and 2.6 g of compound DSC4801 were added into a reaction flask successively at room temperature under the protection of nitrogen, and 8 mL of 0.54 g of chloroethane in tetrahydrofuran was slowly added thereto. After the addition was completed, the above system was heated to reflux and then reacted for 30 minutes. After the reaction was completed, the system was cooled to 0-10° C., filtered and dried to obtain a crude product. The crude product was re-crystallized using a methanol/acetone mixed solvent to obtain 1.33 g of compound DSC4815, with a yield of 43%, [M+H]⁺=376.03;

### Synthesis of Compound DSC4816

1 g of compound DSC4815 and 5 mL of anhydrous ethanol were added into a reaction flask and heated at 50° C., then 0.45 g of silver acetate was added thereto, the reaction mixture was stirred and reacted for 3 h and then subjected to hot filtration, the filtrate was cooled to about 10° C., and then 13 mL of methyl tert-butyl ether was added for crystallization, 0.39 g of compound DSC4816 was obtained, with a yield of 37%, [M+H]⁺=376.03, [M−H]⁻=59.01.

### Example 7: Synthesis of DSC4821

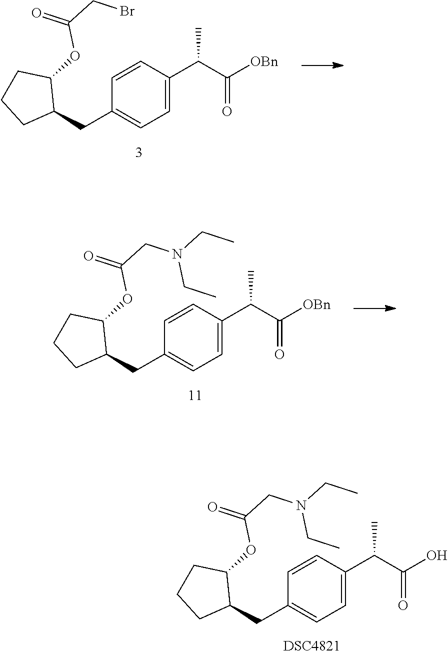 [SMILES: C[C@H](C(=O)OCc1ccccc1)c1ccc(C[C@H]2CCC[C@@H]2OC(=O)CBr)cc1 ; CCN(CC)CC(=O)O[C@H]1CCC[C@@H]1Cc1ccc([C@H](C)C(=O)OCc2ccccc2)cc1 ; CCN(CC)CC(=O)O[C@H]1CCC[C@@H]1Cc1ccc([C@H](C)C(=O)O)cc1]

### Synthesis of Compound 11

0.18 g of compound diethylamine hydrochloride, 0.5 g of compound 3 and 15 mL of anhydrous tetrahydrofuran were added into a reaction flask, the system was cooled to 0° C., and 0.7 mL of triethylamine was added, and then the reaction mixture was heated to 35° C. to react for 4 h. The system was concentrated to dryness and cooled to room temperature, and 15 mL of dichloromethane and 15 mL of water were added into the above system for liquid separation, the organic phase was concentrated to dryness, and then purified via a silica gel column to obtain 0.34 g of compound 11, with a yield of 69%; [M+H]⁺=452.34;

### Synthesis of Compound DSC4821

50 mg of palladium/carbon (10%) was added into 0.3 g of compound 11, 10 mL of methanol was added into the above system, and then the system was connected with a hydrogen balloon. The system was heated to 30° C. to react for 8 h. The system was filtered, and the filtrate was concentrated to dryness, and then purified via a silica gel column to obtain 0.2 g of compound DSC4821, with a yield of 84%; [M−H]⁻=360.29, H NMR (300 MHz, CDCl₃) δ:0.99 (t, 6H), 1.22-1.30 (m, 1H), 1.37-1.66 (m, 3H), 1.45 (d, 3H), 1.70-1.79 (m, 1H), 1.83-2.02 (m, 2H), 2.43-2.66 (m, 6H), 2.75 (dd, 1H), 3.15 (s, 2H), 3.65 (q, 1H), 3.76-3.88 (m, 1H), 7.08-7.27 (m, 4H).

### Example 8: Synthesis of DSC4826

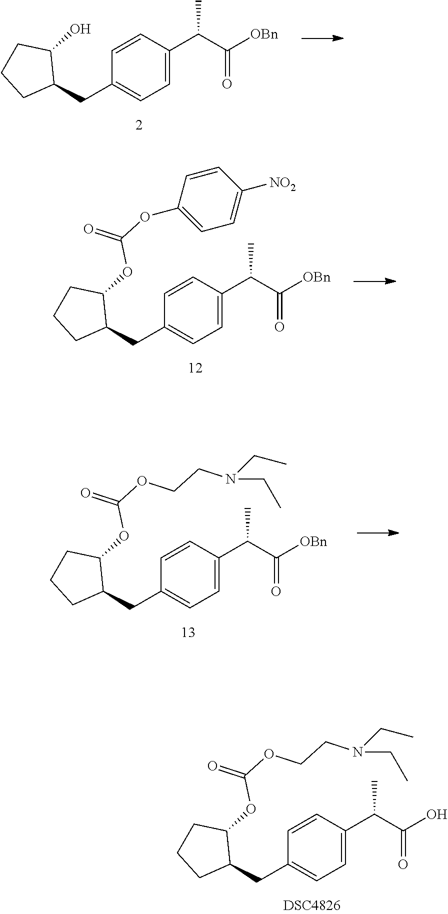 [SMILES: C[C@H](C(=O)OCc1ccccc1)c1ccc(C[C@H]2CCC[C@@H]2O)cc1 ; C[C@H](C(=O)OCc1ccccc1)c1ccc(C[C@H]2CCC[C@@H]2OC(=O)Oc2ccc([N+](=O)[O-])cc2)cc1 ; CCN(CC)CCOC(=O)O[C@H]1CCC[C@@H]1Cc1ccc([C@H](C)C(=O)OCc2ccccc2)cc1 ; CCN(CC)CCOC(=O)O[C@H]1CCC[C@@H]1Cc1ccc([C@H](C)C(=O)O)cc1]

### Synthesis of Compound 12

0.7 g of compound 2, 1.25 g of p-nitrophenyl chloroformate, 1 mL of triethylamine and 10 mL of tetrahydrofuran were added into a reaction flask. The above system was reacted for 1 h at room temperature. The system was concentrated to dryness, then ethyl acetate and water were added for extraction, the organic phase was concentrated to dryness, and then purified via a silica gel column to obtain 0.8 g of compound 12, with a yield 77%, [M+H]⁺=504.21;

### Synthesis of Compound 13

0.13 g of compound diethylaminoethanol and 15 mL of anhydrous tetrahydrofuran were added into a reaction flask, the above system was cooled to 0° C., 65 mg of sodium hydride (60%) was added, and then the system was stirred for 30 min. 0.55 g of compound 12 was added, the reaction mixture was heated to 35° C. to react for 4 h, the above system was concentrated to dryness and then cooled to room temperature, then 15 mL of dichloromethane and 15 mL of water were added for liquid separation, the organic phase was concentrated to dryness, and then purified via a silica gel column to obtain 0.37 g of compound 13, with a yield of 71%; [M+H]⁺=482.39;

### Synthesis of Compound DSC4826

0.18 g of compound DSC4826 was synthesized by using compound 13 as a raw material with reference to the synthesis method of compound DSC4821, with a yield of 84%, [M−H]=390.33, ¹H NMR (300 MHz, CDCl₃) δ:1.01 (t, 6H), 1.22-1.30 (m, 1H), 1.38-1.82 (m, 7H), 1.91-2.06 (m, 2H), 2.46 (dd, 1H), 2.51-2.55 (m, 4H), 2.75-2.88 (m, 3H), 3.71 (q, 1H), 3.86-3.91 (m, 1H), 4.23-4.25 (t, 2H), 7.05-7.20 (m, 4H).

The following example compounds were synthesized according to the same method as those in the above examples by using commercially available compounds or intermediate compounds appropriately synthesized from commercially available compounds.

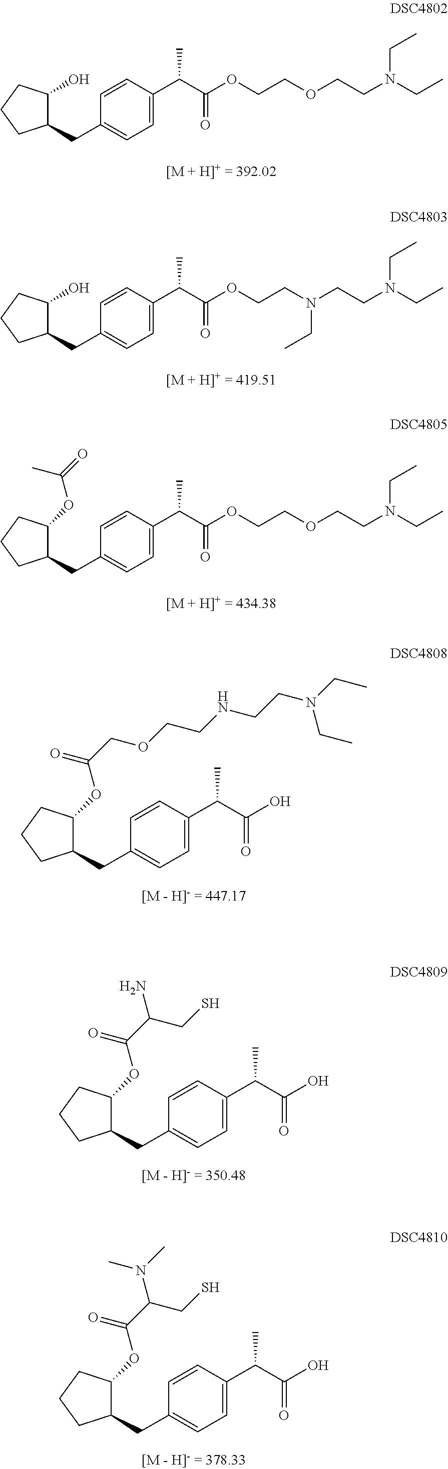 [SMILES: CCN(CC)CCOCCOC(=O)[C@@H](C)c1ccc(C[C@H]2CCC[C@@H]2O)cc1 ; CCN(CC)CCN(CC)CCOC(=O)[C@@H](C)c1ccc(C[C@H]2CCC[C@@H]2O)cc1 ; CCN(CC)CCOCCOC(=O)[C@@H](C)c1ccc(C[C@H]2CCC[C@@H]2OC(C)=O)cc1 ; CCN(CC)CCNCCOCC(=O)O[C@H]1CCC[C@@H]1Cc1ccc([C@H](C)C(=O)O)cc1 ; C[C@H](C(=O)O)c1ccc(C[C@H]2CCC[C@@H]2OC(=O)C(N)CS)cc1 ; C[C@H](C(=O)O)c1ccc(C[C@H]2CCC[C@@H]2OC(=O)C(CS)N(C)C)cc1]

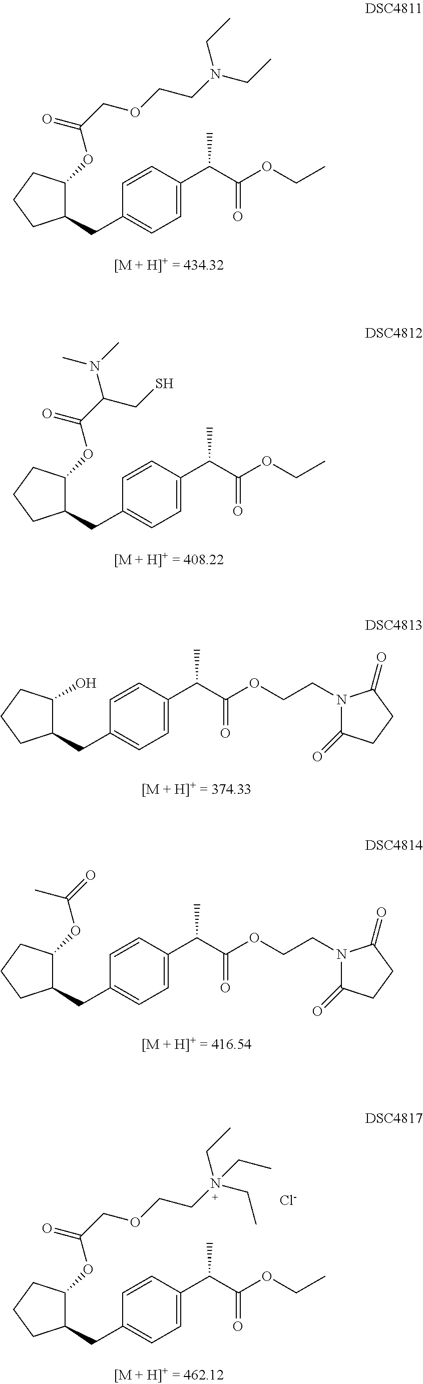

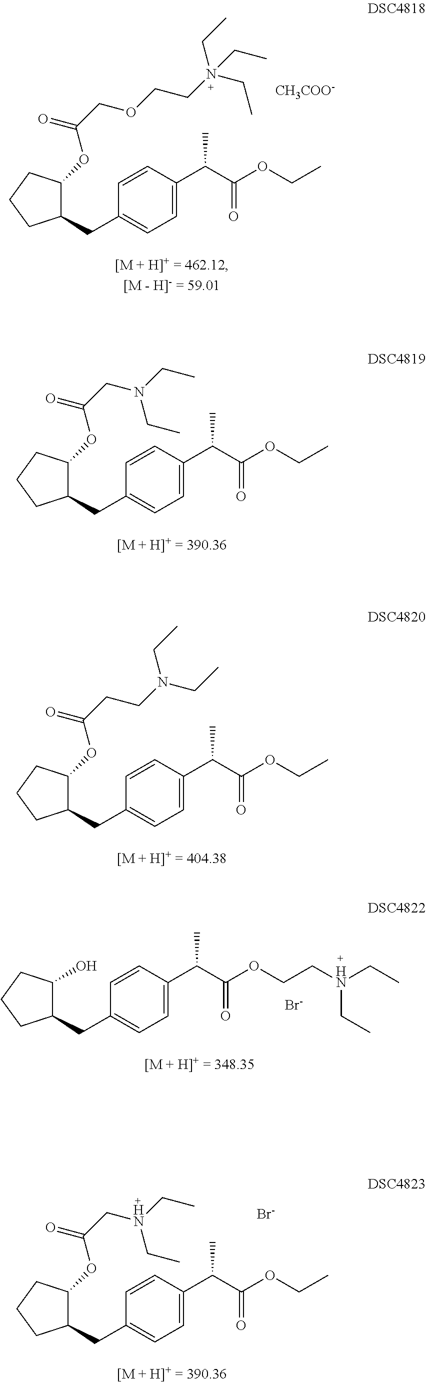

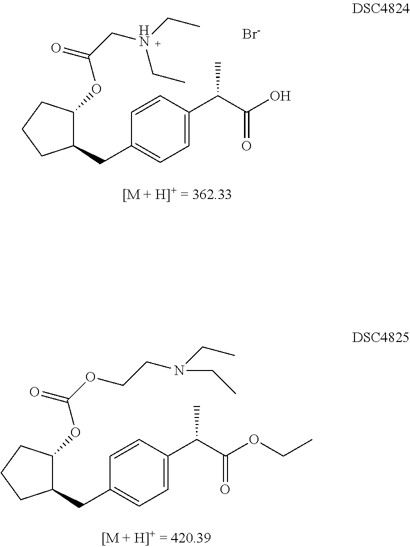

### Synthesis of Comparative Examples

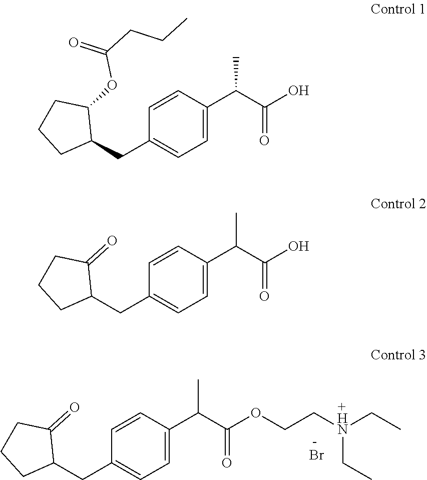

The synthesis of control 1 was achieved with reference to the synthesis methods of example compounds. [M+H]⁻=317.37, ¹H NMR (300 MHz, CDCl₃) δ:0.78 (t, 3H), 1.06-1.29 (m, 3H), 1.38-1.76 (m, 7H), 1.81-2.02 (m, 2H), 2.11 (t, 2H), 2.47 (dd, 1H), 2.79 (dd, 1H), 3.63 (q, 1H), 3.85-3.95 (m, 1H), 7.13-7.27 (m, 4H).

Control 2 was commercially available.

The synthesis of control 3 was synthesized with reference to the synthesis methods in examples of this patent and patent CN103705496B.

### Example 9: Effect on HFLS-RA Cell Proliferation Activity

Grouping and sample concentration: the experiment was divided into blank control groups (2 groups in total, including group 1 and group 2, where group 1 was used for CCK-8 detection at 0 h, and group 2 was used for CCK-8 detection at 72 h; both of the 2 groups did not contain test samples and TNF-α), TNF-α groups (only TNF-α was added without test samples, and the final concentration of TNF-α was 10 ng/mL), test sample groups (test samples were DSC4801-DSC4826 and controls 1 to 3, the structures of controls 1 to 3 can make reference to synthesis parts in comparative examples, and the final concentrations of various test samples for cell incubation and of TNT-α were 100 g/mL and 10 ng/mL, respectively) and positive control groups (the final concentrations of methotrexate for cell incubation and of TNT-α were 1 g/mL and 10 ng/mL, respectively).

Co-incubation: HFLS-RA cells in a logarithmic growth phase were inoculated in a 96-well culture plate at a density of 3×10⁴ cells/ml with an inoculation volume of each well being 100 μL, and cultured for 24 h in a 37° C. and 5% CO₂ incubator (the cell adhesion convergence degree reached about 25%) after inoculation, and then a proper amount of sample solutions was added according to the grouping and final concentrations of samples, with 3 duplicated wells set in each group. Before sampling (0 h), group 1 in blank control group was subjected to CCK-8 detection, and the rest groups were cultured for 72 h in an incubator and then subjected to CCK-8 detection.

Detection and calculation: the culture solution was discarded, and 100 μL of culture medium containing a 10% CCK-8 solution was added into each well and incubated for 2 h at 37° C. The light absorption value (OD value) was measured at 450 nm using a microplate reader, and the cell proliferation rate was calculated according to the following formula. The results are as shown in Table 1.

cell proliferation rate=[OD_{(72 h)}−OD_{(0 h)}]/OD_{(0 h)}×100%, Calculation formula

where, OD_{(0 h)} represents the OD value at 0 h, and OD_{(0 h)} represents the OD value at 72 h.

TABLE 1
HFLS-RA cell proliferation rate (x̅ ± s) 72 h after treatment

| | Cell | | Cell | | Cell |
|---|---|---|---|---|---|
| | proliferation | | proliferation | | proliferation |
| Group | rate | Group | rate | Group | rate |
| Blank control | 61.32 ± 2.01 | TNF-α | 88.57 ± 3.66 | Negative | 18.37 ± 1.32 |
| group | | group | | control group | |
| DSC4801 | 31.18 ± 1.82 | DSC4802 | 27.26 ± 2.41 | DSC4803 | 30.17 ± 1.85 |
| DSC4804 | 29.36 ± 2.33 | DSC4805 | 29.28 ± 1.92 | DSC4806 | 24.19 ± 2.24 |
| DSC4807 | 22.23 ± 3.04 | DSC4808 | 23.11 ± 2.25 | DSC4809 | 22.47 ± 1.99 |
| DSC4810 | 24.95 ± 2.65 | DSC4811 | 22.37 ± 1.78 | DSC4812 | 23.81 ± 2.21 |
| DSC4813 | 22.34 ± 2.72 | DSC4814 | 24.36 ± 1.99 | DSC4815 | 28.31 ± 2.26 |
| DSC4816 | 29.71 ± 2.83 | DSC4817 | 23.32 ± 2.46 | DSC4818 | 22.54 ± 2.14 |
| DSC4819 | 20.36 ± 2.10 | DSC4820 | 21.11 ± 3.56 | DSC4821 | 20.96 ± 1.85 |
| DSC4822 | 31.54 ± 2.23 | DSC4823 | 22.78 ± 1.87 | DSC4824 | 22.17 ± 2.29 |
| DSC4825 | 22.53 ± 2.66 | DSC4826 | 21.43 ± 1.94 | Control 1 | 39.33 ± 3.46 |
| Control 2 | 48.25 ± 3.21 | Control 3 | 44.31 ± 4.15 | | |

It can be seen from Table 1 that compared with blank control group, TNF-α group significantly promotes HFLS-RA cell proliferation (P<0.001); compared with TNF-α group, both of positive control group and test sample group (compounds DSC4801-DSC4826 of the present invention, and controls 1 to 3) significantly inhibit TNF-α induced HFLS-RA cell proliferation (P<0.001); compared with control 1 group, control 2 group and control 3 group, the compound of the present invention has significantly higher inhibition level to the TNF-α induced HFLS-RA cell proliferation (P<0.001). The results show that the compound of the present invention can exert the anti-inflammatory effect through inhibition of HFLS-RA cell proliferation, and is obviously superior to control compounds 1, 2 and 3.

### Example 10: Effect on HFLS-RA Cytokine Secretion

The experiment was divided into a blank control group (containing no test samples or TNF-α), a TNF-α group (the final concentration of TNF-α for cell incubation was 10 ng/mL), test sample groups (8 groups in total, including DSC4801 group, DSC4807 group, DSC4810 group, DSC4813 group, DSC4821 group and DSC4826 group as well as control 1 group, control 3 group, the final concentrations of test samples for cell incubation and of TNT-α were 100 g/mL and 10 ng/mL, respectively).

HFLS-RA cells in a logarithmic growth phase were inoculated in a 6-well culture plate at a density of 5×10⁴ cell/ml with an inoculation volume of each well being 200 μL, and cultured for 2 h in a 37° C. and 5% CO₂ incubator after inoculation, and then a proper amount of sample solutions was added according to the grouping and the final concentrations of samples, with 3 duplicated wells set in each group. Cell supernatant was collected after further culture of 48 h, and the contents of IL-6 and IL-8 in the supernatant were detected using an ELISA kit. The results are as shown in Table 2.

TABLE 2
Effect (x̅ ± s) of samples on HFLS-RA cytokines IL-6 and IL-8 expression

| Group | IL-6 | IL-8 | Group | IL-6 | IL-8 |
|---|---|---|---|---|---|
| Blank control | 44.51 ± 0.58 | 107.35 ± 3.01 | TNF-α | 93.85 ± 1.74 | 162.08 ± 7.35 |
| group | | | group | | |
| DSC4801 | 46.26 ± 0.35 | 119.08 ± 4.55 | DSC4807 | 48.01 ± 0.77 | 121.66 ± 4.78 |
| DSC4810 | 47.88 ± 0.72 | 118.57 ± 3.92 | DSC4813 | 46.82 ± 0.63 | 119.48 ± 4.07 |
| DSC4821 | 46.58 ± 0.61 | 117.72 ± 3.43 | DSC4826 | 47.59 ± 0.45 | 117.77 ± 4.23 |
| Control 1 | 67.45 ± 2.68 | 131.21 ± 6.88 | Control 3 | 74.02 ± 3.53 | 135.76 ± 7.61 |

It can be seen from Table 2 that compared with blank control group, TNF-α can significantly induce the expression of HFLS-RA cell IL-6 and IL-8 inflammatory factors (P<0.001); compared with TNF-α group, control 1 group and control 3 group, the compound of the present invention can significantly inhibit the expression of TNF-α induced HFLS-RA cell IL-6 and IL-8 inflammatory factors (P<0.001). The results show that the compound of the present invention can exert anti-inflammatory effect by inhibiting the expression of IL-6 and IL-8 inflammatory factors, and is obviously superior to control compounds 1 and 3.

### Example 11: Joint Tissue Distribution

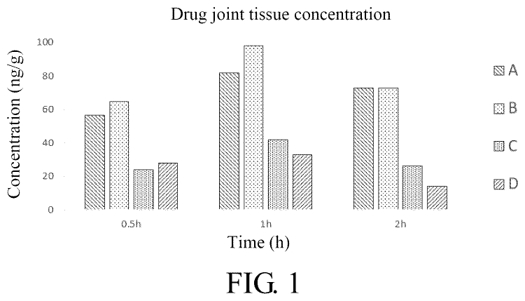

36 healthy male SD rats with a body weight of 200±20 g per rat were fed under conditions of a room temperature of 20-26° C., a humidity of 40%-70% and a light and dark cycle of 12 h/12 h, the rats were fed at liberty in the process of feeding. After adaptive feeding for 3 days, the SD rats were randomly divided into 4 groups (A/B/C/D groups), with 9 rats per group, each group was further randomly divided into 3 time groups, with 3 rats per time group. A suspension (the test samples were suspended with 1% MS, respectively) of the test sample was given to each group via oral gavage, wherein group A was administrated with 3 mg/kg of DSC4813, group B was administrated with 2.94 mg/kg of DSC4821, group C was administrated with 2.47 mg/kg of control 1, and group D was administrated with 2 mg/kg of control 2 (loxoprofen). 3 rats were anesthetized and euthanized in each group at 0.5 h, 1 h and 2 h, respectively after administration. The left and right ankle joints of hind legs were dissected, and the skin at the joints was peeled off. The surrounding tissues of the ankle joints (including inner and outer collateral ligaments, inner and outer triangular ligaments, fascia, tendons, joint synovium, etc.) were weighed. The joint tissue samples were ultrasonically extracted with 10 mL of methanol for 20 min after being cut into pieces, after the extraction was completed, the joint tissue samples were put in EP tubes, sealed and stored at −20° C. for later use. To-be-detected substances were detected by LC-MS/MS (compound trans-OH metabolite was detected for groups A/B/C, loxoprofen and all isomers whose carbonyl was reduced to OH for group D, and the results of group D were a sum of concentrations of loxoprofen and all isomers whose carbonyl was reduced to OH). The results are as shown in FIG. 1.

It can be seen from FIG. 1 that compared with control 1 and control 2, the distribution of the compounds DSC4813 and DSC4821 of the present invention at joints is significantly improved after oral gavage administration (P<0.001). Therefore, it is inferred that the compounds DSC4813 and DSC4821 of the present invention have strong an anti-arthritis effect, and are superior to control compounds 1 and 2.

Although the present invention has been disclosed in preferred embodiments as described above, it is not intended to limit the present invention. Any person skilled in the art may make slight modifications and improvements without departing from the spirit and scope of the present invention. Therefore, the scope of protection of the present invention shall be determined by the appended claims.

1. A chiral aryl propionic acid derivative as shown in formula (I), and a tautomer, a solvate or a pharmaceutically acceptable salt thereof: 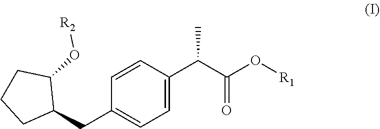
in the formula (I), R₁ is H, or is selected from 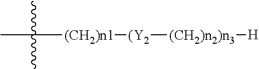
substituted by one or more 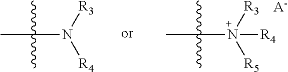
or unsubstituted 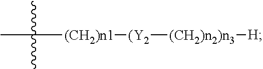
R₂ is H, or is selected from 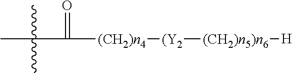
substituted by one or more 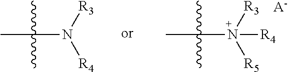
or unsubstituted 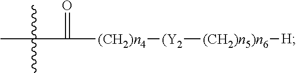
and when R₁ is H, R₂ is not H;
Y₂ is O, or N(R₆), or S;
n₁, n₂, n₃, n₄, n₅ and n₆ are each independently 0, 1, 2, 3, 4, 5, 6, 7, 8, 9, and 10;
R₃, R₄ and R₅ are each independently H, C1-C20 hydrocarbyl, or C1-C20 alkyl carbonyl, or R₃ and R₄ form a ring together with a nitrogen atom to which they are attached;
R₆ is H, or C1-C20 hydrocarbyl; and
A⁻ represents an acceptable inorganic or organic anion.

2. The chiral aryl propionic acid derivative, and a tautomer, a solvate or a pharmaceutically acceptable salt thereof according to claim 1, wherein R₁ is H, and R₂ is 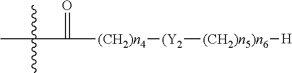
substituted by one or more 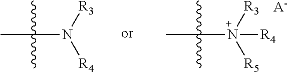
or unsubstituted 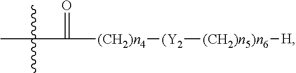
wherein n₄, n₅ and n₆ are each independently 0, 1, 2, 3, 4, 5, 6, 7, 8, 9, and 10.

3. The chiral aryl propionic acid derivative, and a tautomer, a solvate or a pharmaceutically acceptable salt thereof according to claim 1, wherein R₁ is H, and R₂ is 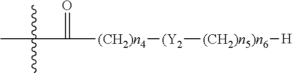
substituted by one or more 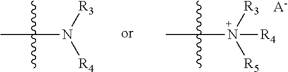
or unsubstituted 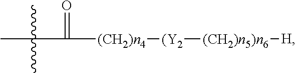
wherein n₄, n₅ and n₆ are each independently 0, 1, 2, 3, and 4.

4. The chiral aryl propionic acid derivative, and a tautomer, a solvate or a pharmaceutically acceptable salt thereof according to claim 1, wherein R₂ is H, and R₁ is 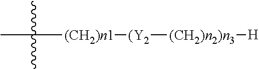
substituted by one or more 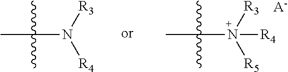
or unsubstituted 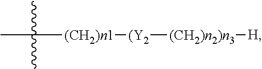
wherein n₁, n₂ and n₃ are each independently 0, 1, 2, 3, 4, 5, 6, 7, 8, 9, and 10.

5. The chiral aryl propionic acid derivative, and a tautomer, a solvate or a pharmaceutically acceptable salt thereof according to claim 1, wherein R₂ is H, and R₁ is 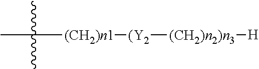
substituted by one or more 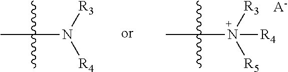
or unsubstituted 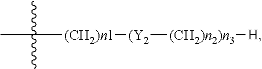
wherein n₁, n₂ and n₃ are each independently 0, 1, 2, 3, and 4.

6. The chiral aryl propionic acid derivative, and a tautomer, a solvate or a pharmaceutically acceptable salt thereof according to claim 1, wherein R₃, R₄ and R₅ are each independently H, or C1-C4 alkyl hydrocarbyl.

7. The chiral aryl propionic acid derivative according to claim 1, which is selected from the following compounds: 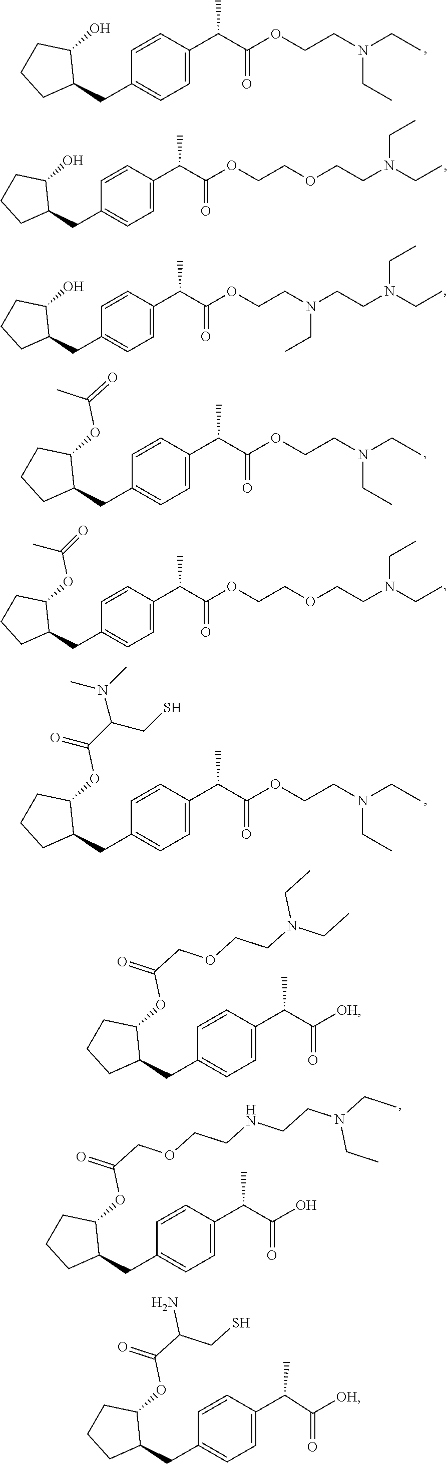 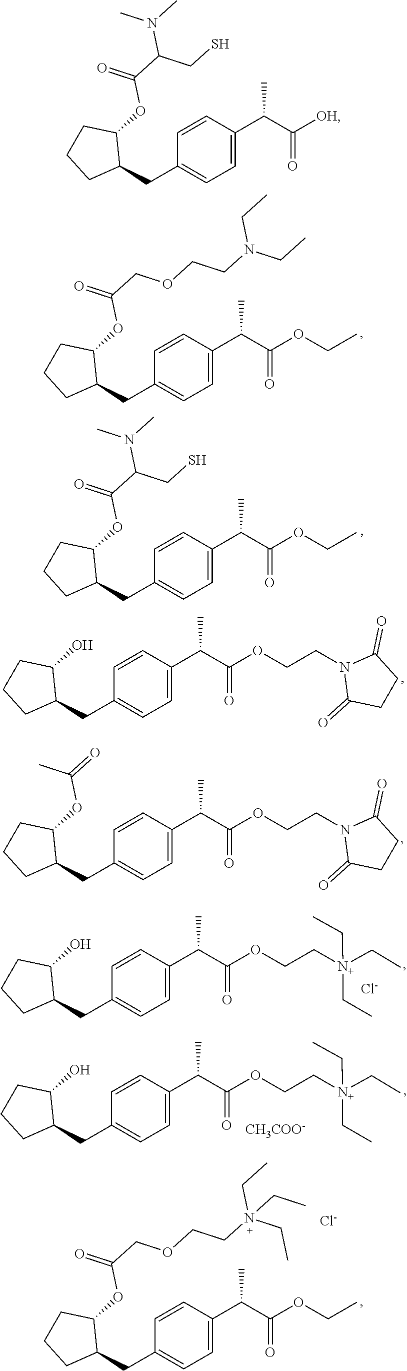 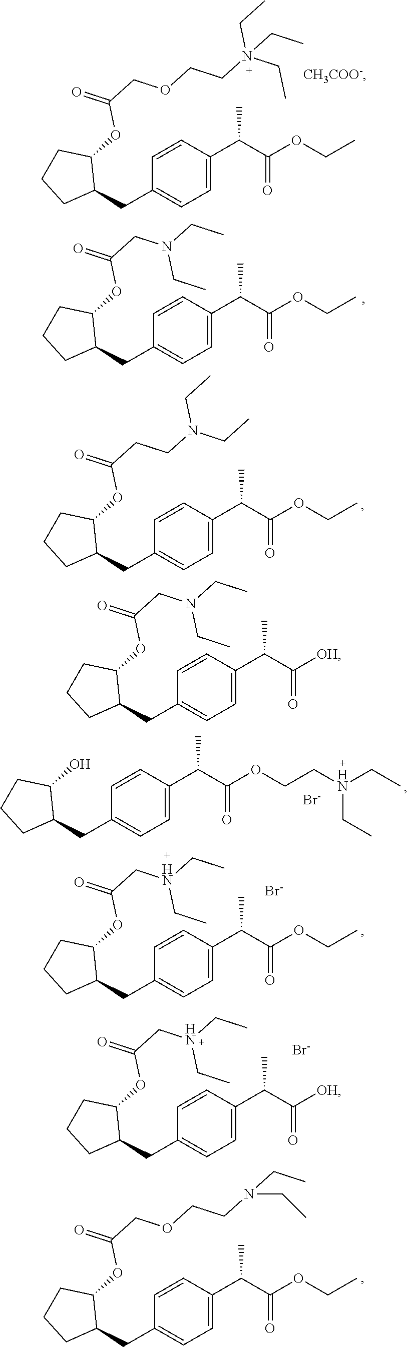 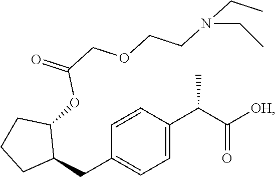 [SMILES: CCN(CC)CCOCC(=O)O[C@H]1CCC[C@@H]1Cc1ccc([C@H](C)C(=O)O)cc1]
or a pharmaceutically acceptable salt thereof.

8. The chiral aryl propionic acid derivative according to claim 1, wherein the pharmaceutically acceptable salt comprises salts formed by the chiral aryl propionic acid derivative and anions, and the anions include but are not limited to fluoride ions, chloride ions, bromide ions, iodide ions, acetate ions, benzoate ions, citrate ions, tartrate ions, oxalate ions, malate ions, ascorbate ions, and fumarate ions, etc.

9. The chiral aryl propionic acid derivative according to claim 1, wherein the pharmaceutically acceptable salt comprises inner salts or intermolecular salts.

10. A pharmaceutical composition comprising the chiral aryl propionic acid derivative, and a tautomer, a solvate or a pharmaceutically acceptable salt thereof according to claim 1.

11. A method comprising manufacturing an antipyretic, analgesic or anti-inflammatory drug that includes the chiral aryl propionic acid derivative, and a tautomer, a solvate or a pharmaceutically acceptable salt thereof according to claim 1.

12. A method comprising manufacturing an antipyretic, analgesic or anti-inflammatory drug that includes the pharmaceutical composition according to claim 10.
